(19) United States
(12) Patent Application Publication
(10) Pub. No.: US 2010/0190186 A1
(43) Pub. Date: Jul. 29, 2010

(54) Method for Pathogen Capture from Mammalian Blood

(76) Inventor: Jian Han, Huntsville, AL (US)

Correspondence Address: DONNA J. RUSSELL, 1492 ANTHONY WAY, MT. JULIET, TN, 37122, US

(21) Appl. No.: 12/685,627

(22) Filed: Jan. 11, 2010

Related U.S. Application Data
(60) Provisional application No. 61/143,811, filed on Jan. 11, 2009.

(51) Int. Cl. G01N 33/53

(52) U.S. Cl. 435/7.2

(57) ABSTRACT

Disclosed is a method for isolating, and thereby concentrating, microorganisms from mammalian blood. The method may be used in conjunction with PCR amplification of the DNA/RNA of the microorganism(s) to identify pathogenic microorganisms within the blood without requiring that those organisms be cultured as a first step in the identification process.

This application claims the benefit of priority of U.S. Provisional Patent Application No. 61/143,811, filed Jan. 11, 2009.

### FIELD OF THE INVENTION

The invention relates to methods for isolating microbes from human clinical samples.

### BACKGROUND OF THE INVENTION

The current gold standard for diagnosis of many microbial diseases, including sepsis, is the culture method. Typically, blood samples are inoculated into culture bottles and incubated to allow any microbes in the sample to reproduce. There are disadvantages to the use of culture methods to accomplish microbial diagnosis, however. Culture does not provide specific identification of an individual genus and/or species of microorganism. After positive culture results are obtained, subcultures and one or more series of biochemical reactions are required to identify any pathogens and evaluate potential drug sensitivity of those pathogens. Sensitivity may be impacted by a patient's exposure to antibiotics prior to sampling. Pathogens may therefore fail to grow in culture, giving false negative results. Furthermore, culture may take hours, days, or even weeks, depending on the genus and species of microorganism, while timely treatment may mean the difference between life and death for the patient.

Molecular diagnosis is generally a more specific and sensitive means to identify microorganisms, requiring significantly less time. However, direct detection of pathogens from a blood sample is still very difficult. There are two major problems with the direct molecular approach for identifying blood-borne pathogens. First, pathogens are usually present in blood samples at very low titer. For example, a bacterial infection may be present when there are as few as 10 copies per milliliter of blood. Furthermore, the nucleic acids (DNA and RNA) of white blood cells contribute to high background for molecular amplification and detection procedures.

What are needed are faster, more sensitive methods for isolating pathogenic microorganisms from mammalian blood specimens.

### SUMMARY OF THE INVENTION

The present invention relates to a method for capturing microbial pathogens from a mammalian blood sample, the method comprising, consisting essentially of, and/or consisting of the steps of admixing with a sample of peripheral blood from a mammalian subject a labeled antibody to a complement protein (i.e., anti-complement antibody), the antibody having appropriate specificity to bind one or more complement proteins attached to one or more pathogens in the blood sample, and capturing the labeled antibody and its associated bound one or more complement proteins attached to one or more pathogens by admixing it with an agent that binds to the label of the labeled anti-complement antibody.

In some aspects, the antibody may be an anti-C3b antibody. In some aspects, the label may be biotin. In some aspects, the agent that binds the label may be avidin or a less- or non-glycosylated compound such as, for example, streptavidin.

### DETAILED DESCRIPTION

The inventors have developed a method for extraction of microbial cells from mammalian blood, which may be used in conjunction with amplification methods such as those described in WO 2005/038039A2, WO 2009/124293A1, and US20090253183A1 to J. Han, which are incorporated herein by reference. Methods such as the amplicon-rescue multiplex polymerase chain reaction (“ARM-PCR”) may be used to amplify the microbial DNA and utilize it to detect the presence of pathogenic microbes in a blood specimen. Using the method, it is possible to accomplish identification of pathogenic microorganisms in a mammalian blood specimen within a matter of hours, where it has previously taken days—and sometimes weeks—to perform identification procedures. While the blood may contain sufficient numbers of microorganisms to signal that there is an active, disease-causing infection, there have not been sufficient numbers isolated from clinical specimens to be readily detected and identified. Therefore, it has previously been common to obtain a clinical sample and try to increase the number of microbial cells by culturing them, so that they will be more readily identifiable.

Briefly, the method involves adding to a peripheral blood sample at least one labeled antibody to a complement protein that binds to a microorganism present in mammalian blood (i.e., an anti-complement antibody), the antibody having appropriate specificity to bind one or more complement proteins attached to one or more pathogens in the blood sample, and capturing the labeled antibody and its associated bound complement/pathogen with an agent that binds the label of the labeled anti-complement protein antibody.

For example, one aspect of the invention involves the steps of obtaining a 10-30 cubic centimeter (cc) sample of peripheral blood or serum from a mammalian subject, admixing biotin-labeled anti-C3b antibodies (1-100 μg) with the blood or serum sample, incubating the anti-C3b antibodies with the sample to allow the antibodies to complex with the C3b bound to any microbial cells that may be present in the blood sample, admixing with the blood/antibody an effective amount of streptavidin-coated magnetic beads to capture the biotin-labeled antibodies which have complexed with C3b coating the microbial cells, removing the blood, and collecting the beads into a small volume of buffer. In the step of admixing the C3b-specific antibody, antibody to complement proteins C5b, C6, C7, C8 and/or C9 antibodies may optionally be added, as well.

The step of admixing labeled antibody with the blood may also be performed by drawing a peripheral blood specimen into a container such as, for example, a Vacutainer® to which labeled antibodies have already been added.

The method utilizes reagents that may readily be obtained from commercial sources. For example, biotin-labeled antibodies may be purchased from a number of vendors (e.g., KPI., Inc., Gaithersburg, Md.; Santa Cruz Biotechnology, Inc., Santa Cruz, Calif.), and the technology for preparing biotin-labeled antibodies in the laboratory has been previously described in the literature (Yolken, R. H., et al, “Enzyme immunoassays for the detection of bacterial antigens utilizing biotin-labeled antibody and peroxidase biotin-avidin complex,” J. Immunol. Methods 1983; 56(3):319-27). Methods for preparing avidin-coated beads have been previously described (Muroi, M., et al., J. Biol. Chem. 2002, 277: 42372-42379), and avidin (or streptavidin) coated magnetic beads are available from a number of sources, including the NanoLink™ and MagnaLink™ product from SoluLink (San Diego, Calif.) and Dynabeads® from Invitrogen Corporation (Carlsbad, Calif.). Pierce Chemical (Rockford, Ill.) also produces a kit for biotinylating proteins (EZ-Link®).

In this description of the invention, reference will be most often made to C3b antibodies. It is to be understood, however, that antibodies to other binding proteins of the complement system that may bind, and therefore “tag,” microorganisms within the mammalian blood are also within the scope of the invention (e.g., anti-C5b, and C6, C7, C8 and C9 antibodies).

Magnetic beads are generally added at a quantity of about 50,000 to about 500,000 beads per sample, this range being intended to encompass sub-ranges (e.g., about 50,000 to about 100,000; about 100,000 to about 250,000, etc.) within this range. A magnet placed at or near an outside surface of the sample tube may be used to concentrate the beads on an inside surface of the sample tube. Once capture of the beads is accomplished, the blood may be removed and the beads washed with phosphate buffered saline (PBS). Bacteria bound to the beads may then be collected in a small amount (e.g., 100 microliters) of PBS. DNA and/or RNA may then be extracted using an appropriate extraction method known to those of skill in the art. The amount of DNA/RNA is typically quite low, so the nucleic acid may be amplified using an amplicon-rescue multiplex PCR (ARM-PCR) method previously described by inventor Jian Han in US2009025318A1 and WO 2009/124293A1. Briefly, ARM-PCR is a method for producing detectable amounts of target polynucleotides from a clinical, environmental, or food sample, the method comprising amplifying, in a first amplification reaction, one or more target polynucleotides to produce amplicons using nested primers, at least a portion of the nested primers comprising additional nucleotides to incorporate into a resulting amplicon a binding site for a communal primer; separating the amplicons from the first amplification reaction from one or more unused primers from the first amplification reaction; and amplifying, by the addition of communal primers in a second amplification reaction, the amplicons of the first amplification reaction having at least one binding site for a communal primer.

In one aspect of the present invention, a human peripheral blood specimen comprising 10-30 cc of blood is collected. Anti-C3b antibodies are added to and combined with the blood and the antibody/blood combination is incubated at 37° C. for 10-30 min to allow antibodies to bind to C3b on the pathogen surface. Magnetic beads are added at 50,000-150,000 beads per sample, followed by an additional incubation period of 10-30 minutes. A magnet is applied to concentrate beads on the wall of the sample tube. The blood is removed and the beads washed by adding and removing 500 μl, and this wash step is repeated: The bead-bound bacteria are then collected in a small amount (e.g., 100 μl) of PBS buffer. DNA and/or RNA is extracted using standard extraction methods known to those of skill in the art, and the DNA and/or RNA is then utilized in the ARM-PCR multiplex amplification procedure to produce sufficient quantities of microbial DNA to be readily detected by standard means. In one aspect, Luminex® technology may be used for detection.

The method utilizes pathogen-binding proteins of the mammalian complement system (part of the innate immune system), already present in mammalian blood, as an intermediate to capture pathogens and concentrate their number in a sample volume. The mammalian innate immune system coats pathogens (bacteria, viral, fungal and parasites) with proteins, such as the complement proteins, in a process called opsonization. This process is usually not specific to particular pathogens, but almost any pathogen may be targeted by complement proteins, facilitating phagocytosis or lymphocyte-mediated killing of the coated pathogen. The C3b protein of the complement system, for example, tags the microorganisms for destruction by phagocytes. In the present method, biotin-labeled antibodies to C3b (i.e., anti-C3b antibodies) allow the capture of C3b-coated pathogens from blood.

In normal human blood, C3 is one of the most abundant proteins. C3 is an enzyme that is split into two fragments by components of either the classical pathway or the alternative pathway. The classical pathway is induced by antibodies, especially IgG and IgM, while the alternative pathway is nonspecifically stimulated by bacterial products such as lipopolysaccharide (LPS). The products of the C3 split include a small peptide, C3a, which is chemotactic for phagocytic immune cells and results in local vasodilation by causing the release of the C5a fragment from the C5 protein. The other part of C3, C3b, coats antigens on the surface of foreign organisms and acts to opsonize or “tag” the organism for destruction. C3b also reacts with other components of the complement system to form a membrane attack complex (MAC) consisting of C5b, C6, C7, C8 and C9.

One aspect of the invention may utilize a biotin-labeled anti-C3b antibody to find the “tagged” pathogens in the blood samples, then, streptavidine-coated magnetic beads may be added into the blood to capture pathogen-bound C3b complexed with biotin-labeled anti-C3b antibody. After washing to remove intact cells and cellular debri, microbial cells are eluted from the beads by heating, or the bead-bound microbial cells are used directly for down-stream nucleic acid purification, amplification, and detection.

Since the complement system is generally common among mammals, the method may be used for the detection of pathogenic microorganisms in humans, primates, dogs, cats, and other animals. Therefore, the invention may have human medical, as well as veterinary, uses.

The invention may be further described by means of the following non-limiting examples.

### EXAMPLE

A 10-30 cc sample of human peripheral blood or serum was collected. Biotin-labeled mouse anti-human C3b antibodies (1-100 μg) were added to the blood. The blood/antibody mixture was incubated at 37° C. for 10-30 min to allow the antibodies to bind to C3b on the pathogen surface. Magnetic beads were added (50,000-150,000 beads) and the mixture was incubated for an additional 10-30 minutes. A magnet was applied to the outside of the tube into which the blood, antibodies, and beads had been placed, and the beads were attracted to the side of the tube, where they were concentrated in the area to which the magnetic force had been applied. The blood was gently removed and the beads were gently washed twice with 500 μl PBS. The beads were collected in 100 μl PBS. DNA was extracted, ARM-PCR was performed, and Luminex® technology was used to isolate and identify Escherichia coli in the blood sample.

What is claimed is:

1. A method for capturing microbial pathogens from mammalian blood samples, the method comprising the steps of
a. admixing with a sample of peripheral blood from a mammalian subject at least one labeled antibody to a complement protein that binds to a microorganism present in mammalian blood, the at least one antibody having appropriate specificity to bind one or more complement proteins attached to one or more microorganisms in the blood sample; and
b. admixing with the labeled antibody and its associated bound complement/microorganism an agent that binds the label of the labeled antibody and thereby facilitates isolation of the microorganism.

2. The method of claim 1 wherein the antibody is an anti-C3b antibody.

3. The method of claim 1 wherein the antibody is labeled with biotin.

4. The method of claim 3 wherein the agent that binds the label of the labeled antibody is chosen from among the group consisting of avidin and streptavidin.
